(19) United States
(12) Patent Application Publication
(10) Pub. No.: US 2012/0040445 A1
(43) Pub. Date: Feb. 16, 2012

(54) GAS-FREE FLUID CHAMBER

(75) Inventors: Peter Hermanus Bouma, Eindhoven (NL); Martinus Leonardus Josephus Geyselaers, Eindhoven (NL)

(73) Assignee: KONINKLIJKE PHILIPS ELECTRONICS N.V., EINDHOVEN (NL)

(21) Appl. No.: 13/264,231

(22) PCT Filed: Apr. 8, 2010
(86) PCT No.: PCT/IB10/51524

§ 371 Date: Oct. 13, 2011

(30) Foreign Application Priority Data
Apr. 15, 2009 (EP) 09157958.1

(51) Int. Cl. C12M 1/34; B65B 3/04; C12M 1/00

(52) U.S. Cl. 435/287.2; 435/289.1; 141/1

(57) ABSTRACT

A Gas-free Fluid chamber for PCR. The present invention relates to a device with a fluid chamber suitable for performing a polymerized chain reaction for gas-free filling. Such devices may be used in the field of e.g. molecular diagnostics.

### FIELD OF THE INVENTION

The present invention relates to a device with a fluid chamber suitable for, for instance, performing a polymerase chain reaction. Such devices may be used in the field of e.g. molecular diagnostics.

### BACKGROUND OF THE INVENTION

In the field of molecular diagnostics, it is nowadays common to use microfluidic devices. Such microfluidic devices or microfluidic systems typically comprise a network of chambers which are connected by channels that provide for communication between the different fluid chambers. The fluid chambers as well as the channels typically have microscale dimensions with, for example, the dimensions of the channels typically being in the range of 0.1 μm to about 1 mm. Such microfluidic devices are described inter alia in U.S. Pat. No. 6,843,281 B1.

A process that is commonly used in the field of molecular diagnostics is the so called polymerase chain reaction (PCR). During this reaction a small amount of liquid (typically 100 μl or less) containing DNA is thermally processed in order to amplify a specific part of the DNA.

To this end a set of primers is added to the liquid comprising the DNA together with enzymes and desoxyribonucleotides (dNTPs). The liquid is then subjected to consecutive steps of denaturing, annealing and elongation. During the denaturing steps, double stranded DNA is separated into single stranded DNA molecules. During the annealing step, primers being specific for a certain portion of the DNA within the liquid hybridise to the segregated single strands. During the elongation step, enzymes such as a DNA polymerase then extend the primers. Typically, the elongation temperature is higher than the annealing temperature and denaturation temperature is higher than the elongation temperature. By running the steps of denaturing, annealing and elongation in subsequent cycles, it is possible to amplify small amounts by the rate of 2ⁿ with n designating the number of cycles and with one cycle comprising a denaturing, annealing and elongation step. The above description refers to the basic principle of PCR, there are numerous specific approaches to allow specific uses of PCR.

One commonly used PCR technology is so called real time fluorescent PCR (rtPCR). This technology refers to the use of differently labelled primers during PCR. Such primers may be provided in a form that, when not annealed to another nucleic acid do not emit any fluorescence but which upon annealing and elongation emit a fluorescent signal after having been excitated with an appropriate wavelength.

This approach therefore allows for online-monitoring of the performance of a PCR reaction and, provided that appropriate calibration and control experiments are run in parallel, even allow for online determination of the concentration of the original concentration of the DNA being present in the sample.

PCR reactions are typically performed in fluid chambers, also called reaction chambers that allow for heating and cooling the fluid chamber at a very fast rate to e.g. the denaturing, annealing and elongation temperature. For the present invention of the term ‘reaction chamber’ is a species of the term ‘fluid chamber’, namely a fluid chamber in which a reaction, for instance PCR, can take place. However, the general idea of the present invention concerns the gas free filling of a fluid chamber, which may be a reaction chamber.

One problem currently encountered during PCR reactions and particularly during online detection of real time PCR is that gas-bubbles such as air are trapped in the fluid chamber.

In view of the dimensions of the fluid chamber, such trapped gas-bubbles may impede the performance of the PCR reactions as well as the (online) detection of the amplified nucleic acid molecules.

Therefore, there is a constant interest in new PCR systems with fluid chambers that allow for gas-free filling in order to improve both PCR efficiency as well as detection of amplified nucleic acid products. There is a general interest in fluid chambers as they may be used in microfluidic devices which allow for gas-free filling.

### SUMMARY OF THE INVENTION

It is one objective of the present invention to provide a fluid chamber which can be used in a microfluidic device and allows for gas-free filling.

It is a further objective of the present invention to provide a fluid chamber that is suitable for PCR and allows for gas-free filling.

These and other objectives as they will become apparent from the ensuing description hereinafter form the subject matter of the independent claim. Some of the preferred embodiments of the present invention form the subject matter of the dependent claims.

The present invention in one embodiment thus relates to a fluid chamber (1) being in communication with,

a first channel (2) suitable for functioning as an inlet for fluids into said fluid chamber;

a second channel (3) suitable for functioning as an outlet for fluids out of the fluid chamber;

wherein at least one protrusion (4) projects into the fluid chamber,

and wherein said protrusion (4) is positioned at the locations where the second channel (3) is connected to the fluid chamber.

In one embodiment the surface of said protrusion (4) inside the fluid chamber (1) is smooth.

Smooth means that a protrusion does not have a sharp corner except for maybe at its basis where it is connected to the wall of the fluid chamber. At a sharp corner the angle with a fluid front is not defined resulting in reduced control of fluid propagation.

A, for instance, semicircular protrusion has the advantage over a rectangular protrusion that an advancing fluid front can follow the smooth surface of the semicircular protrusion easier than in the case of the rectangular protrusion which comprises a sharp edge at which the angle between the fluid front and the protrusion is not well defined.

### Examples of Smooth Shapes are Elliptical and Circular Shapes.

In principle, the fluid chamber may take any three-dimensional form with smoothly curved walls viewed from above.

Thus, it may take a circular or an elliptical cross-sectional form (5) when viewed from above.

Preferably the fluid chamber is of cylindrical form with a circular or elliptical cross-sectional shape (5) when viewed from above.

In one embodiment, the fluid chamber is of cylindrical form (5) with a circular or elliptical cross-sectional shape (5), when viewed from above and the first channel (2) and the second channel (3) are connected to the side walls of the fluid chamber of cylindrical form. The fluid chamber will typically be configured in terms of its dimensions and material to allow for incorporation into a microfluidic device. Preferably, the fluid chamber will be configured to allow for performing a PCR within the fluid chamber.

Thus, in one embodiment, the diameter D of the fluid chamber (1) will be in the range of 100 μm to a couple of cm and the height H of the fluid chamber (1) will be in the range of 100 μm to 1 cm.

The diameter or depth d (7) of the protrusion (4) of circular or elliptical shape which is positioned at the location where the second (outlet) channel (3) is connected to the fluid chamber projects into the fluid chamber by 20 μm to 1 cm. Preferably the diameter d (7) of the protrusion (4) of circular or elliptical shape will typically be in the range of about 50 μm to about 500 μm.

As a general rule, the diameter D (6) of the fluid chamber should be greater than or equal to about 10 times the dimensions of the diameter d (7) of the protrusion. In a preferred embodiment of the invention, the diameter D (6) of the fluid chamber of cylindrical form with a circular or elliptical cross-sectional shape (5), when viewed from above is in the range of 1 mm to 10 mm, the height H is in the range of 0.2 mm to 5 mm and the diameter d (7) is in the range of 0.1 to 1 mm.

The first (inlet) channel (2) and the second (outlet) channel (3) can be positioned at opposite sites of the fluid chamber (1). However, they may also be positioned at any other angle with respect to each other. If the first (inlet) channel (2) and the third (outlet) channel (3) are positioned next o each other (see e.g. FIG. 4), only one extrusion may be necessary.

As mentioned above, the fluid chamber (1) is configured such that it is suitable for performing PCR in the fluid chamber. Thus, in one embodiment the fluid chamber may be in communication, e.g. connected to means for controlling the temperature within the fluid chamber. The temperature control means may thus allow the temperature of a liquid within the fluid chamber to be raised and lowered to temperatures as they are required for the e.g. denaturing, annealing and extension step.

In one embodiment the fluid chamber may be further modified to comprise at least one transparent section. Such a transparent section may allow for online monitoring of the reaction within the fluid chamber. In one embodiment the at least one transparent section within the fluid chamber may allow for online optical monitoring of amplified nucleic acids during rtPCR.

In one embodiment the fluid chamber may be transparent as a whole.

Another embodiment relates due a device such as a cartridge comprising a fluid chamber in accordance with the present invention.

Other embodiments of the present invention will become apparent from the detailed description hereinafter.

### BRIEF DESCRIPTION OF THE DRAWINGS

FIG. 1 depicts a top view of a fluid chamber (1) that is connected to a first channel (2) suitable for functioning as an inlet for fluids into fluid chamber and a second channel (3) suitable for functioning as an outlet for fluids out of the fluid chamber. At the locations where the second channel (3) is connected to the fluid chamber (1), FIG. 1 depicts further the protrusion (4) of circular or elliptical shape that projects into the fluid chamber.

FIG. 2: FIG. 2 a) to i) depict different stages when a fluid chamber of FIG. 1 is filled with liquid. In FIG. 2a, liquid moves through the first (inlet) channel (2). In FIG. 2b, liquid enters into the fluid chamber (1). FIG. 2c) to FIG. 2e) show how liquid asymmetrically projects further into the fluid chamber. In FIG. 2f), the liquid stops at the first protrusion which it encounters. In FIG. 2g) to FIG. 2h), the remaining part of the fluid chamber is filled with liquid until the liquid stops at the second protrusion. In FIG. 2i), the liquid is pushed out of the second (outlet) channel (3).

FIG. 3 depicts a fluid chamber (1) wherein the first (inlet) channel (2) and the second (outlet) channel (3) are not opposite to each other.

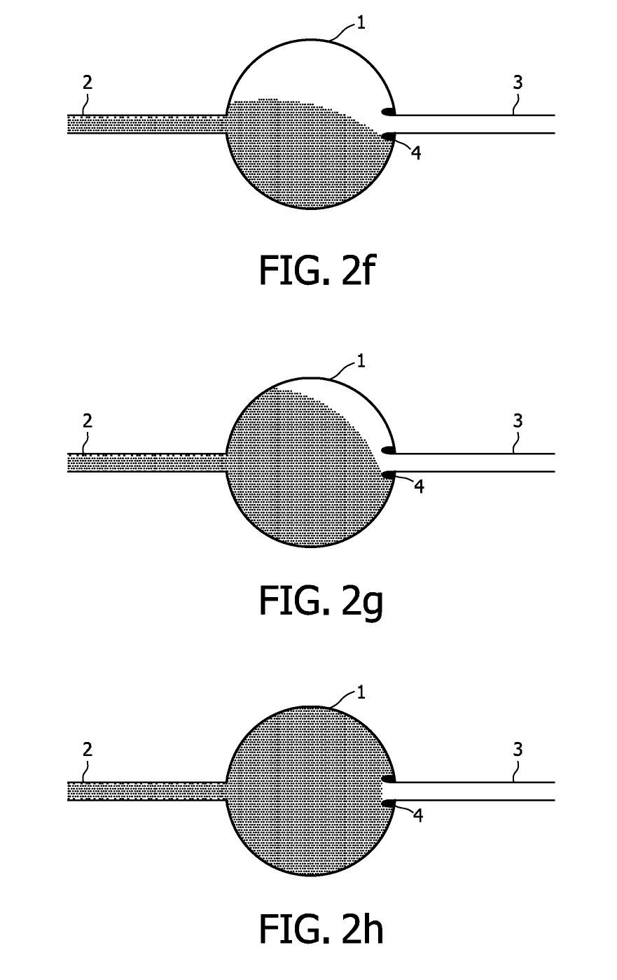

FIG. 4 depicts a fluid chamber (1) wherein the first (inlet) channel (2) and the second (outlet) channel (3) enter and leave the fluid chamber (1) at the same location and wherein the protrusion (4) is located between the first and second channel.

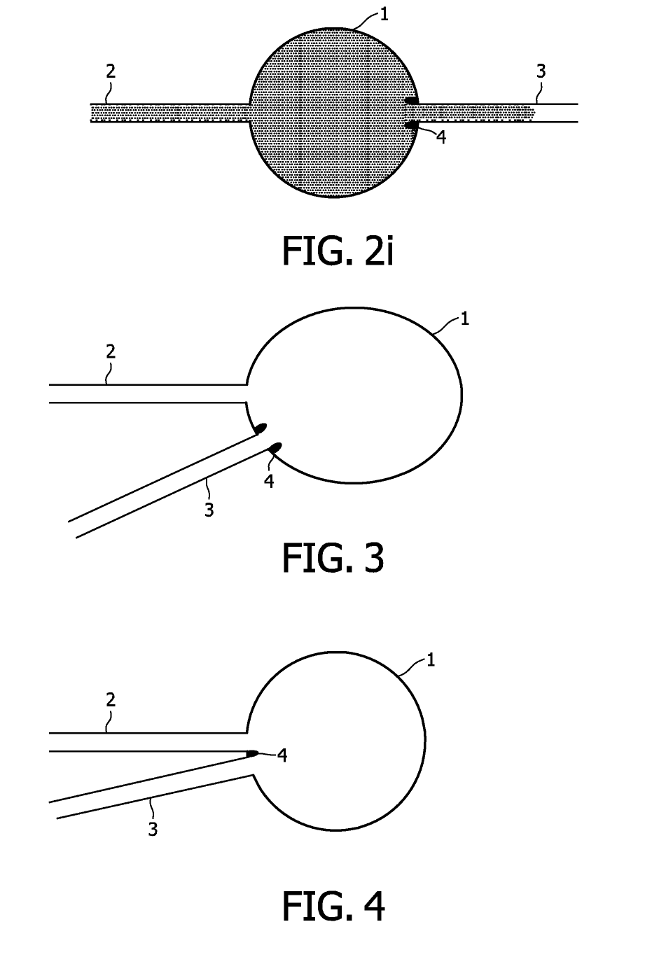

### DETAILED DESCRIPTION OF EMBODIMENTS

It has been found that positioning of a protrusion of circular or elliptical shape at the location where an outlet channel connects to a fluid chamber enables gas-free filling of a fluid chamber.

Before the invention is described in detail with respect to some of its preferred embodiments, the following general definitions are provided.

The present invention as illustratively described in the following may suitably be practiced in the absence of any element or elements, limitation or limitations, not specifically disclosed herein.

The present invention will be described with respect to particular embodiments and with reference to certain drawings but the invention is not limited thereto but only by the claims. The drawings as described are only schematic and non-limiting. In the drawings, the size of some of the elements may be exaggerated and not drawn on scale for illustrative purposes.

Where the term “comprising” is used in the present description and claims, it does not exclude other elements. For the purposes of the present invention, the term “consisting of” is considered to be a preferred embodiment of the term “comprising of”. If hereinafter a group is defined to comprise at least a certain number of embodiments, this is also to be understood to disclose a group which preferably consists only of these embodiments.

Where an indefinite or definite article is used when referring to a singular noun, e.g. “a”, “an” or “the”, this includes a plural of that noun unless something else is specifically stated. The terms “about” or “approximately” in the context of the present invention denote an interval of accuracy that the person skilled in the art will understand to still ensure the technical effect of the feature in question. The term typically indicates deviation from the indicated numerical value of ±10%, and preferably of ±5%.

Further definitions of terms will be given in the following in the context of which the terms are used.

As mentioned above, the present invention in one embodiment relates to a fluid chamber (1) being in communication with,

a first channel (2) suitable for functioning as an inlet for fluids into said fluid chamber;

a second channel (3) suitable for functioning as an outlet for fluids out of the fluid chamber;

wherein at least one protrusion (4) projects into the fluid chamber; and

wherein said at least one protrusion (4) is positioned at the locations where the second channel (3) is connected to the fluid chamber.

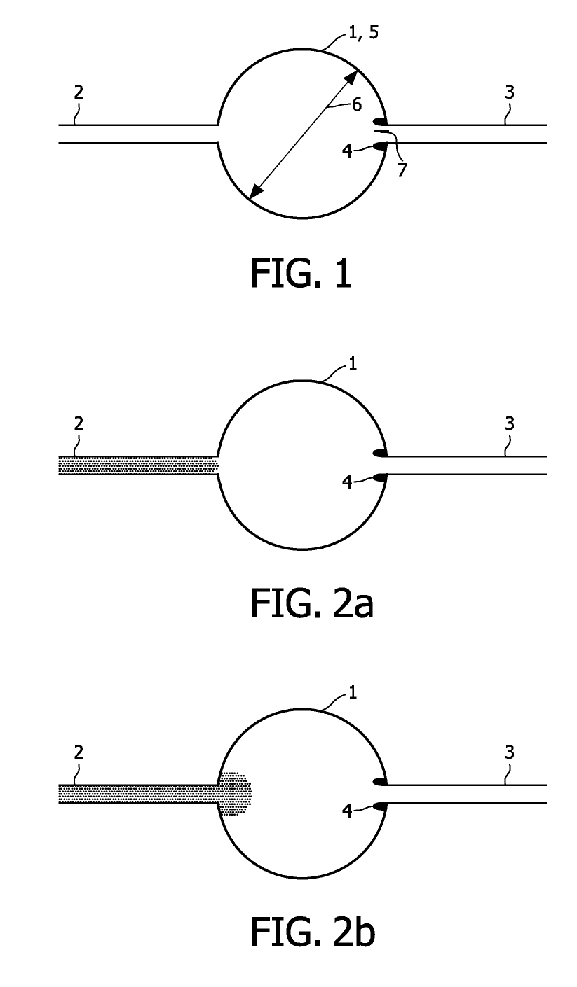

The principle underlying the present invention is depicted in FIG. 1. FIG. 1 shows a fluid chamber viewed from the top. The fluid chamber (1) has a circular cross-sectional shape (5) when viewed from above and is connected to a first channel (2) and a second channel (3).

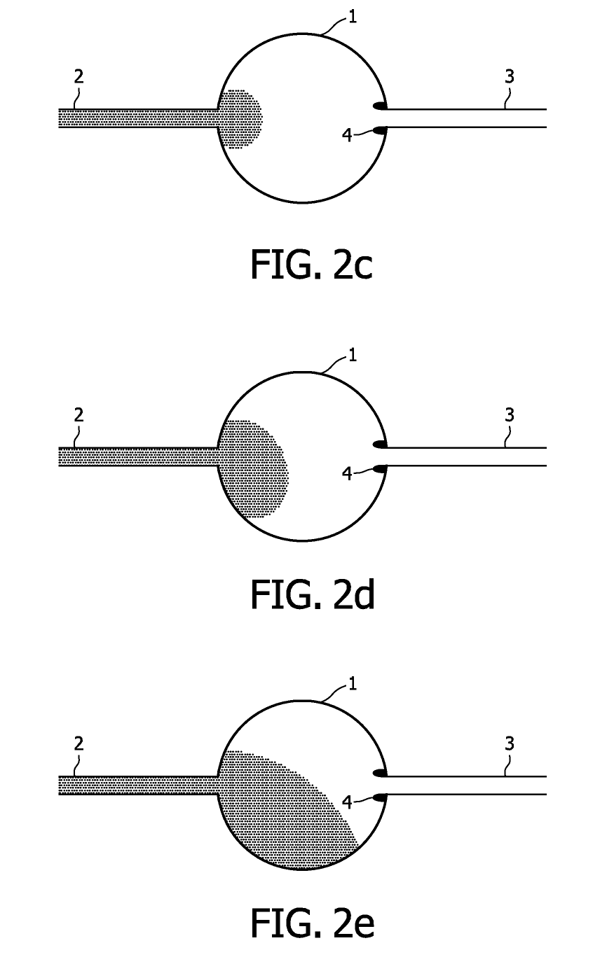

When the chamber is partially filled with liquid during the liquid filling process (as depicted in FIG. 2b) to FIG. 2e) the position of the liquid-gas interface is quite often not determined due to rotational symmetry of the chamber. Thus, liquid is present on the left side of this interface and gas on the right side. The shape of this interface depends on the contact angle between the interface and the solid wall.

As shown in FIG. 1, at the position where the second channel (3) enters the fluid chamber, a protrusion (4) of circular shape projects into the fluid chamber. This protrusion of circular or elliptical shape which may also be designated as a protrusion of half cylindrical shape is typically small compared to the other dimensions of the chamber. When the liquid-gas interface reaches one of these protrusion structures, then the propagation of the interface will temporarily stop there until the interface reaches also the protrusion structure on the other side of the channel (see FIG. 20 to FIG. 2h). By this process most if not all of the gas will be driven out of the fluid chamber and the liquid flows into the channel (3) functioning as an outlet channel. This process is depicted in FIG. 2 and FIG. 5

In general, a fluid chamber of the above mentioned embodiment can take any form. Preferably, such a fluid chamber when viewed from the top may have a cross-sectional circular form or an elliptical form (5).

It is preferred for the fluid chambers of the present invention to have a cylindrical form with a cross-sectional circular or elliptical form when viewed from above.

The diameter D (6) of the fluid chamber (1) will be in the range of 100 μm to a couple of cm. Preferably, D (6) will be in the range of about 100 μm to about 10 cm, of about 200 μm to about 9 cm, of about 300 μm to about 8 cm, of about 400 μm to about 7 cm, of about 500 μm to about 6 cm, of about 600 μm to about 5 cm, of about 700 μm to about 4 cm, of about 800 μm to about 3 cm, of about 900 μm to about 2 cm, of about 1 mm to about 1 cm such as about preferably 0.2 mm, about preferably 0.3 mm, about preferably 0.4 mm, about preferably 0.5 mm, about preferably 0.6 mm, about preferably 0.7 mm, about preferably 0.8 mm or about preferably 0.9 mm.

The height H of the fluid chamber (1) will typically be in the range of about 100 μm to about 1 cm, of about 200 μm to about 9 mm, of about 300 μm to about 8 mm, of about 400 μm to about 7 mm, of about 500 μm to about 6 mm, of about 600 μm to about 5 mm, of about 700 μm to about 4 mm, of about 800 μm to about 3 mm, of about 900 μm to about 2 mm or of preferably about 1 mm.

The term “diameter” D (6) as far as it relates to cylindrical fluid chambers of cross-sectional circular shape, is used in its common sense form. As far as the term “diameter” refers to cylindrical fluid chambers with a cross-sectional elliptical shape, it refers to the major axis of an ellipse.

As already mentioned above, the protrusion of circular or elliptical shape (4) is typically smaller than the diameter of the fluid chamber. Typically the diameter d (7) of the protrusion of circular or elliptical shape is smaller than the diameter of the fluid chamber by a factor of equal to or at least about 10, such as at least about 15, at least about 20 or preferably at least about 25.

The diameter or depth d (7) of the at least one protrusion (4) of circular or elliptical shape which is positioned at the location where the second (outlet) channel (3) is connected to the fluid chamber projects into the fluid chamber by about 20 μm to about 1 cm. Preferably the diameter d (7) of the protrusion (4) of circular or elliptical shape will typically be in the range of about 30 μm to about 1 mm, of about 40 μm to about 900 μm, of about 50 μm to about 800 μm, of about 60 μm to about 700 μm, of about 70 μm to about 600 μm, of about 80 μm to about 500 μm, of about 90 μm to about 300 μm, such preferably about 100 μm or about 200 μm.

In a preferred embodiment of the invention, the diameter D (6) of the fluid chamber of cylindrical form with a circular or elliptical cross-sectional shape (5), when viewed from above is in the range of 1 mm to 10 mm such as 5 mm, the height H is in the range of 0.2 mm to 2 mm such as 1 mm and the diameter d (7) is in the range of 0.1 to 0.5 mm such as 200 μm.

The term “diameter” d (7) in the context of the protrusion is commonly used as it refers to a protrusion of circular shape. As far as a protrusion of elliptical shape is concerned, the term refers to the major axis.

Typically, the fluid chambers according to the present invention may have internal volumes of about 1 μl to about 200 microliters with volumes of about 10 to about 100 micro litres such as 25 microliters being preferred.

The channels being connected to the fluid chamber will typically have a diameter of about 10 μm to about 5 mm such as about 100 μm to about 500 μm. The channels may have any form such as round form or a rectangular form. In the case where a non-round form is used, the aforementioned dimensions may refer to e.g. the width and height of a rectangular channel. Thus the width may be e.g. 500 μm and the height may be 100 μm.

Further, in one embodiment, fluid chambers in accordance with the present invention may be configured such that they are suitable for performing PCR within the fluid chamber. Thus, the fluid chamber may be connected to temperature control elements such as heating and cooling elements as they are typically used in micro fluidic devices to allow performance of PCR reactions.

Further, in one preferred embodiment the fluid chambers in accordance with the present invention may comprise at least one transparent section. Such a transparent section may e.g. be positioned in the top of the fluid chamber to allow for optical detection of the reaction products that are formed within the fluid chamber. In a typical embodiment a transparent section may be used that allows for online optical monitoring of a rtPCR reaction going on within the fluid chamber.

Typically, the fluid chamber will be made from materials that are suitable to withstand the conditions that are required for the reaction being performed within the fluid chamber. In the case of a PCR reaction one will thus select materials as they are commonly used for PCR fluid chambers. Such materials may include e.g. polymers, plastics, resins, metals including metal alloys, metal oxides, inorganic glasses etc. as long as the contact angle between liquid and surface is larger than 90 degrees (i.e hydrophobic for water) Particular polymeric materials may include for example polyethylene, polypropylene, such as high-density polypropylene, polytetrafluoroethylene, polymethylmethacrylate, polycarbonate, polyethyleneteraphthalate, polystyrene and styrene etc. Polypropylene may be preferred.

The transparent section if it is e.g. used for detecting a rtPCR reaction may e.g. be made from a transparent hydrophobic material, for instance polypropylene.

The present invention further relates to a method of substantially completely filling a fluid chamber with a liquid comprising at least the following steps:

a. Providing a fluid chamber as described above;
b. Introducing a liquid into the first channel (2) of a fluid chamber as described above;
c. Filling the fluid chamber such that the liquid leaves the filled fluid chamber through the second channel (2) of the fluid chamber as described above.

The term “substantially completely” means that the fluid chamber is filled with liquid without having gas bubbles in the fluid chamber.

Similarly, the invention relates to the use of a fluid chamber as described above for gas-free filling with a liquid.

The present invention has been described with respect to some specific embodiments which are however not to be construed as being limiting.

### REFERENCE NUMBERS

(1) fluid chamber
(2) first channel suitable as an inlet
(3) second channel suitable as an outlet
(4) protrusion into fluid chamber which is positioned at second channel
(5) cross-sectional circular or elliptical shape of fluid chamber when viewed from above
(6) diameter D of fluid chamber
(7) diameter d of protrusion

1. A fluid chamber (1) being in communication with,
a first channel (2) suitable for functioning as an inlet for fluids into said fluid chamber;
a second channel (3) suitable for functioning as an outlet for fluids out of the fluid chamber;
wherein at least one protrusion (4) projects into the fluid chamber,
and wherein said protrusion (4) is positioned at the locations where the second channel (3) is connected to the fluid chamber.

2. A fluid chamber (1) wherein the surface of said protrusion (4) inside the fluid chamber (1) is smooth.

3. A fluid chamber (1) according to claim 2, wherein the protrusion (4) is of circular or elliptical shape.

4. A fluid chamber (1) according to claim 1, wherein the fluid chamber is of cylindrical form with a circular or elliptical cross-sectional shape (5), when viewed from above; and
wherein the first channel (2) and the second channel (3) are connected to the side walls of fluid chamber of cylindrical form.

5. A fluid chamber according to claim 1, wherein the diameter (6) of the fluid chamber (1) is in the range of about 100 μm to about 10 cm and wherein the height of the fluid chamber is in the range of about 100 μm to about 1 cm.

6. A fluid chamber according to claim 1, wherein the diameter (7) of the protrusion (4) of circular or elliptical shape is smaller than the diameter (6) of the fluid chamber (1) by a factor of equal to or at least about 10.

7. A fluid chamber according to claim 1, wherein the diameter (7) of the protrusion (4) of circular or elliptical shape is in the range of about 10 μm to about 1 cm.

8. A fluid chamber according to claim 1, wherein the fluid chamber (1) is configured such that it is suitable for performing polymerase chain reactions in the fluid chamber.

9. A fluid chamber according to claim 1, wherein means for controlling the temperature within the fluid chamber are in communication with the fluid chamber.

10. A fluid chamber according to claim 1, wherein the fluid chamber comprises at least one transparent section.

11. A fluid chamber according to claim 1, wherein the fluid chamber is made from polypropylene.

12. Use of a fluid chamber according to claim 1 for gas-free filling with a liquid.

13. Method of completely filling a fluid chamber with a liquid comprising at least the following steps:
a. Providing a fluid chamber according to claim 1;
b. Introducing a liquid into the first channel (2) of a fluid chamber according to claim 1.

14. Device comprising a fluid chamber of claim 1.

15. Device of claim 14 wherein the device is a cartridge.
